(19) United States
(12) Patent Application Publication
(10) Pub. No.: US 2003/0211496 A1
(43) Pub. Date: Nov. 13, 2003

(54) Quality control reagents for nucleic acid microarrays

(76) Inventors: Robert C Getts, Collegeville, PA (US); James M Kadushin, Glibertsville, PA (US); Ari Zak, New Rochelle, NY (US)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/239,961

(22) PCT Filed: Mar. 29, 2001
(86) PCT No.: PCT/US01/10328

(30) Foreign Application Priority Data
Mar. 29, 2000 (US) 60192896

(51) Int. Cl. C12Q 1/68; C12M 1/34

(52) U.S. Cl. 435/6; 435/287.2

(57) ABSTRACT

Disclosed are reagents for conducting quality control reactions on microarrays of nucleic acids and kits containing the reagents, along with directions for conducting the reactions with the components in the kits. Also disclosed are methods of preparing the kits as well as using them to conduct the quality control reactions. A preferred reagent embodiment is illustrated in FIG. 3.

### TECHNICAL FIELD

The present invention relates to nucleic acid hybridization methodologies, and more particularly to quality control reagents used in the course of conducting such methods.

### BACKGROUND OF THE INVENTION

Technology relating to genetic analysis has substantially evolved over the past two decades, and particularly during the last 10 years. The state of the art entails the preparation of microarrays of hundreds, thousands or in some cases, hundreds of thousands of oligonucleotides or clones of DNA sequences of interest e.g., genes or portions thereof implicated in human disease such as cancers, Alzheimer's, etc. Formerly, the DNA molecules were cloned in cells such as bacteria to generate sufficient quantities to prepare the microarray. The advent of PCR technology provided a much easier way to generate a large quantity of DNA. Thus, rather than making copies of an entire vector, the DNA of interest is flanked by primer sequences such as T7, T3, M13 forward, M13 reverse and SP6. The PCR reaction results in amplification of the DNA and the flanking sequences. Once the DNA is amplified or the oligonucleotides synthesized, it is spotted onto the microarray. Microarrays are available commercially or may be customized by an individual laboratory, depending upon the specific DNAs or diagnostic application of interest.

A DNA microarray can function properly only if each DNA probe spotted onto the coated slide is firmly attached to the slide and available for hybridization to the labeled sample. Verification that each feature is functioning properly is vital to the subsequent quantitation and analysis of the data. Thus, to enhance the precision and reliability of diagnoses made based upon nucleic acid hybridization, the microarrays are typically subjected to one or more types of quality control. In general, these involve staining with a fluorescent dye such as ethidium bromide or using single fluroescently-labeled oligonucleotides. Quality control specific to the microarray is a two-pronged issue, namely: (1) has DNA been placed on the position on the microarray; and (2) is it the DNA that was intended. Current quality control methods are regarded as deficient in one or more respects because there is a lack of functional testing for hybridization and limited sensitivity.

Accordingly, there is a need for quality control reagents to test DNA microarrays from these standpoints.

### SUMMARY OF THE INVENTION

A first aspect of the present invention is directed to a kit for conducting quality control reactions on a microarray of nucleic acids. The kits contains the following elements:

a container containing a first buffer solution comprising a first reagent containing a nucleic acid matrix carrying a detectable label, the matrix having attached thereto an oligonucleotide probe that binds nucleic acid contained on the microarray; and

directions for conducting the quality control reactions with said first reagent and the nucleic acids on the microarray.

In preferred embodiments, the matrix contains a polynucleotide monomer having an intermediate region containing a linear, double stranded waist region having a first end and a second end, wherein the first end terminates with two single stranded hybridization regions, each from one strand of the waist region, and the second end terminates with one or two single stranded hybridization regions, each from one strand of the waist region. More preferably, each of the hybridization regions and the waist region of the monomer contains sequences obtained from a master sequence containing no repeats of subsequences having from 2 to 6 nucleotides. In other preferred embodiments, the matrix contains a plurality of such polynucleotide monomers bonded together by hybridization at at least one such hybridization region.

The oligonucleotide probe is attached to the matrix via ligation or hybridization and cross-linking. It may be designed with a random sequence, in which case, it is advantageously used as a qualitative reagent in the case that it will detect the presence of nucleic acid on the microarray. In other embodiments, the oligonucleotide has a sequence substantially complementary to a known nucleic acid sequence that is supposed to be present on the microarray. Thus, in preferred embodiments, the oligonucleotide binds a primer sequence such as T7, T3, M13 forward, M13 reverse or SP6.

Preferred detectable labels are fluorescent dyes such as Cy3™, Cy5™, Alexa™ 488 and Alexa™ 594.

In yet other preferred embodiments, the kit includes a second container containing a second buffer solution for conducting the quality control reactions. The kit may also contain another container containing a second buffer solution containing a second reagent. The second reagent differs from the first reagent in that the detectable label is resolvable from the detectable label on the first reagent and/or the oligonucleotide binds different nucleic acid contained on the microarray. Thus, many different quality control reactions may be conducted substantially simultaneously.

The oligonucleotide probe does not have to be part of the kit. It can be synthesized and attached to the matrix by the end user. Accordingly, a second aspect of the present invention is directed to a kit for conducting quality control reactions on a microarray of nucleic acids, containing a first container containing a first buffer solution containing a nucleic acid matrix carrying a detectable label. The kit also contains directions for (a) producing a reagent by attaching to said matrix an oligonucleotide having a first end portion that attaches to the matrix and a second end portion that binds nucleic acid on the microarray, (which preferably includes the sequence of the outer arm or branch of the matrix to which the first end portion binds) and (b) conducting the quality control reactions with the reagent and the nucleic acids on the microarray. In preferred embodiments, the kit also contains a second container containing a second buffer solution in which to conduct the quality control reactions between the reagent and the nucleic acid on the microarray.

As an alternative to the second aspect, the kit may contain the oligonucleotide probe in a separate container. Thus, in a third aspect, the present invention provides a kit for conducting quality control reactions on a microarray of nucleic acids, including:

a first container containing a first buffer solution containing a nucleic acid matrix carrying a detectable label;

a second container containing a second buffer solution containing an oligonucleotide having a first end portion that attaches to the matrix and a second end portion that binds nucleic acid on the microarray; and

directions for attaching the oligonucleotide to the matrix to prepare the first reagent and for conducting the quality control reactions with the first reagent and the nucleic acids on the microarray.

In preferred embodiments, the oligonucleotide is attached to the matrix indirectly, e.g., via hybridization and cross-linking to a complement capture oligonucleotide that is directly attached to the matrix. The complement capture oligonucleotide may be provided already attached to the matrix, in a separate container of the kit, or synthesized and attached to the matrix by the end user. The kit may further include a third container containing a third buffer solution in which to attach the oligonucleotide probe to the matrix. Methods for preparing the kits are also provided.

A further aspect of the present invention is directed to a method for conducting quality control reactions on a microarray of nucleic acids. The method entails:

providing the microarray of nucleic acids;

providing a reagent comprising a nucleic acid matrix carrying a detectable label, said matrix having attached thereto an oligonucleotide that binds nucleic acid contained on the microarray;

contacting the reagent with the microarray; and

detecting the label as an indication of the presence or type of nucleic acid on the microarray. This aspect of the invention pertains to the kits described above in connection with the first aspect of the present invention.

Yet a further aspect of the present invention is directed to a method for conducting quality control reactions on a microarray of nucleic acids. This method entails:

providing the microarray of nucleic acids;

providing a nucleic acid matrix carrying a detectable label, and attaching to the matrix an oligonucleotide having a first end portion that attaches to the matrix and a second end portion that binds nucleic acid on the microarray;

contacting the reagent with the microarray; and

detecting the label as an indication of the presence or type of nucleic acid on the microarray. This aspect pertains to the use of the kits described in the second and third aspects of the present invention. In preferred embodiments, directions for conducting the reactions are also provided.

### BRIEF DESCRIPTION OF THE DRAWINGS

FIGS. 1A and 1B are schematic representation of elements of the quality control reagents useful in the present invention;

FIGS. 2 and 3 are schematic representations of quality control reagents of the present invention; and

FIG. 4 is a flow diagram that schematically illustrates a method for conducting quality control reactions of the present invention.

### BEST MODE OF CARRYING OUT THE INVENTION

The present invention is directed to quality control reagents for use with nucleic acid microarrays, kits containing the reagents, and methods for preparing and using the quality control reagents and kits.

Thus, one aspect of the present invention is directed to the quality control reagents and their intermediates. One element of the reagent is a labeled moiety that contains a branched matrix composed of individual oligonucleotides or nucleic acid-like molecules (a polynucleotide matrix) that carries a plurality of detectable labels. In one embodiment, the labeled moiety is attached to a randomer. In another embodiment, it is attached to a DNA sequence that hybridizes with a specific primer sequence. Yet another embodiment is directed to an intermediate for the preparation of a quality control reagent, and contains the labeled moiety attached to a bridging oligonucleotide. A free end of the bridging oligonucleotide serves as a point of attachment for an oligonucleotide i.e., a probe that binds the primer sequence and/or any portion of the arrayed DNA under the conditions in which the products are used. The probe oligonucleotide may be provided by or for the end user. ps The Polynucleotide Matrix

A variety of branched nucleic acid matrices designed to carry a plurality of labels are known in the art. See, e.g., U.S. Pat. Nos. 5,124,246 and 5,656,731 to Urdea, et al. Preferred matrices exhibit a relatively highly ordered and symmetrical architecture and are commonly referred to as “nucleic acid matrices”. Dendritic molecules, per se, are highly-branched arborescent structures that were originally assembled from organic polymers. They have found industrial applications as chemical reagents, lubricants, contrast media for magnetic resonance and the like. See, e.g., Barth et al., Bioconjugate Chemistry 5:58-66 (1994); Gitsov & Frechet, Macromolecules 26:6536-6546 (1993); Hawker & Frechet, J. Amer. Chem. Soc. 112:7638-7647 (1990a); Hawker & Frechet, Macromolecules 23:4726-4729 (1990b); Hawker et al., J. Chem. Soc. Perkin Trans. 1:1287-1297 (1993); Lochmann et al. J. Amer. Chem. Soc. 115:7043-7044 (1993); Miller et al., J. Amer. Chem. Soc. 114:1018-1025 (1992); Mousy et al., Macromolecules 25:2401-2406 (1992); Naylor et al., J. Amer. Chem. Soc. 111:2339-2341 (1989); Spindler & Frechet, Macromolecules 26:4809-4813 (1993); Turner et al., Macromolecules 26:4617-4623 (1993); Wiener,. et al., Magnetic Resonance Med. 31(1):1-8 (1994) and U.S. Pat. Nos. 4,558,120; 4,507,466; 4,568,737; 4,587,329; 4,857,599; 5,527,524; and 5,338,532 to Tomalia. Matrices offer several advantages over other molecular architectures. First, they contact the maximum volume or area with a minimum of structural elements. Second, the growth of matrices can be highly controlled to yield molecules of ideal size and molecular weight. Finally, the large number of defined “ends” can be derivatized to yield highly labeled molecules with defined spacing between the labels. Nucleic acid matrices have been constructed following the technology that was originally applied to conventional organic polymers. See Hudson et al., “Nucleic Acid Dendrimers: Novel Biopolymer Structures,” Am. Chem. Soc. 115:2119-2124 (1993); and U.S. Pat. No. 5,561,043 to Cantor.

More preferred are nucleic acid matrices that have some overall similarity to the aforementioned purely dendritic structures but yet are structurally distinct therefrom. These nucleic acid matrices are taught in U.S. Pat. Nos. 5,175,270; 5,484,904 and 5,487,973 to Nilsen et al. The unique molecular design of Nilsen's matrices accommodates a large number of labels, in the order of several hundred, resulting in more than a 100-fold amplification of the signal compared to various prior art methods. Target nucleic acids can be detected even when present in the sample in extremely small (e.g., femptogram (10⁻¹⁵)) amounts.

These polynucleotides are defined in terms of a plurality of polynucleotide monomers bonded together by hybridization; each polynucleotide monomer having an intermediate region comprising a linear, double stranded waist region having a first end and a second end, the first end terminating with two single stranded hybridization regions, each from one strand of the waist region, and the second end terminating with one or two single stranded hybridization regions, each from one strand of the waist region; and in the dendritic polynucleotide each polynucleotide monomer is hybridization bonded to at least one other polynucleotide monomer at at least one such hybridization region. Due to the way in which these matrices are assembled, the outer layer of monomers of the polynucleotide contains a plurality of free hybridization arms. The number of such arms varies depending upon the structure of the individual monomers and the number of monomer layers contained in the polynucleotide. The assembly via hybridization may begin with an initiator nucleic acid molecule having three or more single stranded regions. In these cases, hybridization of nucleic acid molecules to the free single stranded ends of the initiator generates the first layer product. In the case of hybridization of an initiator with three arms with three-armed matrix monomers, a first layer having six arms is produced. The more preferred seven strand dendritic structure utilizes monomers with four arms; consequently, the first layer possesses twelve arms. Subsequent layers of hybridization lead to a geometric expansion of the single-stranded ends and a three-dimensional dendritic organization of nucleic acids.

In even more preferred embodiments, the polynucleotides exhibit maximal self-assembly. In these embodiments, each of said hybridization regions and said waist regions of said plurality of monomers comprise sequences containing no repeats of subsequences having X nucleotides, wherein X is an integer of at least 2. In preferred embodiments, X is an integer from 2 to 6 or 7; in more preferred embodiments, X is 3, 4, 5 or 6. These more preferred matrices are assemblies of several layers of monomers. The labeled moiety may contain just a single monomer, however. See WO 99/06595.

As disclosed herein, the matrices per se may be “nucleic acid-like” in the sense that their composition is not limited strictly to the use of individual nucleotides and nucleic acids. For example, the matrices may be assemblies using peptide-nucleic acids (PNAs) or nucleic acid analogs prepared in accordance with standard techniques.

In its broadest sense, the detectable label is any compound employed as a means for detecting an oligonucleotide. Examples of labels include fluorescent dyes, biotin, digoxigenin, radionucleotides, antibodies, enzymes and receptors such that detection of the labeled polynucleotide (the labeled moiety) is by fluorescence, conjugation to streptavidin and/or avidin, antigen-antibody and/or antibody-antibody interactions, quantitation of radioactivity, and catalytic and/or ligand-receptor interactions. Fluorescent dyes are preferred. Examples include Cy3™ and Cy5™ (both available from Amersham Pharmacia Biotech), fluorescein, FluorX, Oregon Green™, the Alexa™ series dyes (e.g., Alexa™ 488 and 594), and the BODIPY™ series dyes, all of which are commercially available from various sources including NEN, Molecular Probes, Boehringer Mannheim and Amersham Life Sciences.

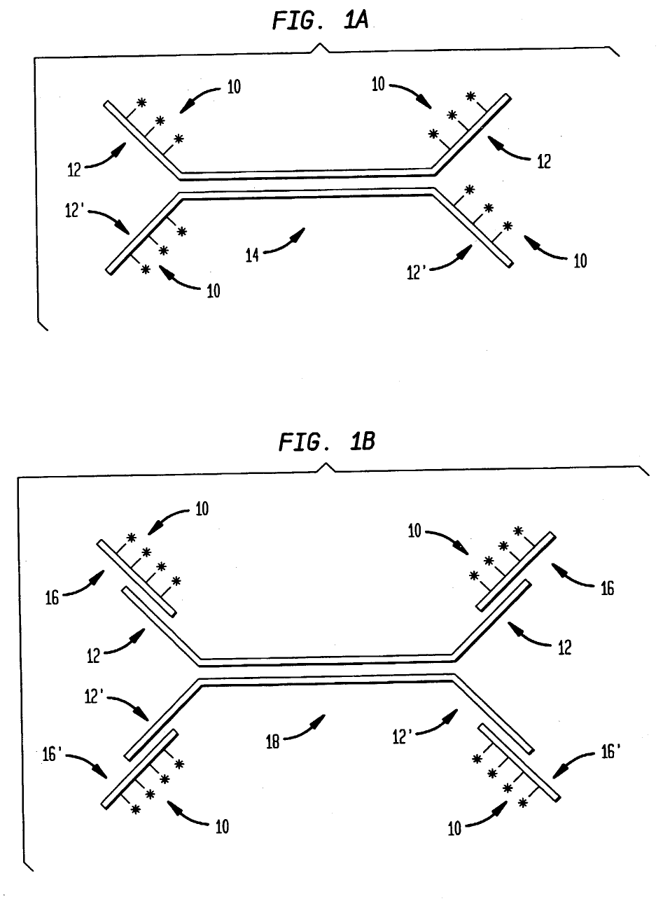

The individual label molecules may be attached to the polynucleotide matrix in several ways. FIGS. 1A and 1B are schematic illustrations of such, wherein the matrix contains a single monomer. As shown in FIG. 1A, label molecules 10 are attached to individual nucleotides of free outer arms 12 and 12′ of matrix 14. FIG. 1B illustrates a preferred embodiment wherein label molecules 10 are attached to individual nucleotide bases of oligonucleotides 16 and 16′ which are hybridized with free, single stranded outer arms 12 and 12′ respectively, of polynucleotide monomeric matrix 18. The oligonucleotide has one end portion that hybridizes with a branch or in the case of the more preferred embodiments, a free outer arm, of the matrix. Such labeled oligonucleotides are described in U.S. Pat. No. 6,046,038. These embodiments allow for enhanced detection capabilities that may or may not be needed in the case of quality control, depending upon the sensitivity of the instrumentation.

In a first preferred embodiment of the present invention, the labeled polynucleotide matrix is directly attached to an oligonucleotide that binds a target on the microarray. The oligo can be attached to a branch or free outer arm of the matrix by direct ligation or via hybridization and cross-linking (for purposes of enhanced stability). The sequence of the target complementary oligo can be relatively random or specific in nature. An oligo containing a random sequence is referred to as a randomer. Generally, the sequence is from about 8 to about 20 bases. Due to the random nature of the sequence, the product serves well as a general quality control reagent because it hybridizes with virtually any DNA molecule under the conditions in which it is used. A binding event between the quality control reagent and a position on the microarray indicates that DNA has been spotted onto a specific position thereon.

Alternatively, the product is relatively “customized” and the target complementary sequence is referred to as a specific complementary sequence. It is attached to the matrix instead of a randomer. It is preferred that the sequence is complementary to the primer sequence(s) that flanks the DNA molecules contained in each of the wells which more often than not, is the same for all positions on the microarray. Examples of oligonucleotides complementary to commonly used primer sequences are set forth below.

Sp6-7BO Oligo

(SEQ ID NO:1)

5′-ATT TAg gTg ACA CTA TAT TTT TCg-3′
T7-7BO Oligo

(SEQ ID NO:2)

5′-TAA TAC gAC TCA CTA TAg ggT TTT TCg-3′
T3-7BO Oligo

(SEQ ID NO:3)

5′-TAA CCC TCA CTA AAg ggA TTT TTC g-3′
M13F-7BO Oligo (M13 FORWARD)

(SEQ ID NO:4)

5′-gTT gTA AAA CgA CCA gTg ttt ttc G-3′
M13R-7BO Oligo (M13 REVERSE)

(SEQ ID NO:5)

5′ CAC ACA ggA AAC AgC TAT gTT TTT Cg-3′

Perfect complementarity for known primers (and other nucleic acids) is the case if for no other reason than the primer sequences are known. In general, however, perfect complementarity is not required. Base mismatches can be accommodated provided that the sequence binds the primer under conditions in which the quality-control reagent is used (in which case the oligonucleotide is said to have a sequence substantially complementary to a nucleic acid believed to be present on the microarray).

The product will be sold in the form of a kit. The quality control reagent is separately contained in an appropriate buffer solution, preferably a neutral buffer. The kit may also contain a hybridization buffer to be used along with the quality control reagent to actually conduct the quality control hybridization reactions with the microarrayed DNA. Other suitable buffers are commercially available e.g., ExpressHyb™ (Clontech), Ultrahyb™ (Ambion). Otherwise, they may be prepared on an individual basis. The kit further contains manufacturer's protocols or directions for use. Two specific protocols, the first directed to a quality-control reagent with a “randomer” sequence and the second directed to a reagent having a sequence specific to a known primer, are set forth below.

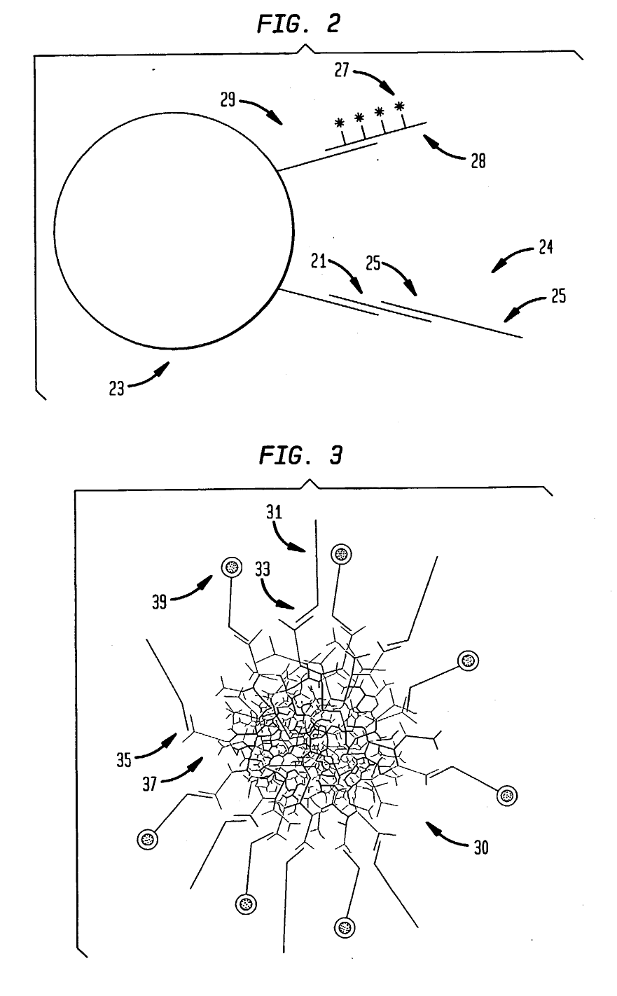

In a second preferred embodiment of the present invention, a free branch or outer arm of the polynucleotide matrix serves as a complement capture oligonucleotide or is attached to a complement capture oligonucleotide (e.g., by direct ligation or by hybridization and cross-linking). FIG. 2 schematically illustrates one such example wherein complement capture oligonucleotide 21 is hybridized with a portion of outer free arm 22 of matrix 23. Oligonucleotide 24 is bifunctional and contains one end portion 25 that functions as a matrix capture sequence and binds to complement capture oligonucleotide 21 or a portion thereof, and another end portion or subsequence 26 that is a randomer or a specific complementary sequence as described above. The matrix-capture sequence is attached to the complement capture sequence or the outer free arm of the matrix, preferably by hybridization and/or cross-linking. Oligonucleotide 24 may be provided along with a suitable buffer in a separate vial of the kit, in which case the kit further contains a buffer in which to conduct the attachment of oligonucleotide 24 to complement capture oligonucleotide 21. Alternatively, the end user may prepare oligonucleotide 24 and attach it to the matrix, in which case, the protocol or directions further contain the sequence of at least the portion or subsequence of complement capture oligonucleotide 21 to which end portion 25 attaches. Thus, use of this embodiment of the present invention entails attaching the bifunctional oligonucleotide to the matrix (e.g., via an outer free arm or indirectly via a complement capture oligonucleotide) and then contacting the fully assembled labeled matrix with the target sequences present on the microarray. In FIG. 2, labels 27 are attached to the matrix by oligonucleotide 28 that hybridizes with free outer arm 29.

Plainly, modifications with respect to the components in the kit and the procedures for using the components are well within the skill of the routineer in the art. For instance, the kit may contain, in separate containers, two or more quality control agents each of which carries a label resolvable from the other label(s). The differently labeled reagents may carry the same or different target complementary oligonucleotide. Each individual reagent may have specificity for more than one nucleic acid sequence believed to be present on the microarray (e.g., by having attached oligos that bind nucleic acids having different sequences). Likewise, in the second preferred embodiment, the kit may contain two or more types of B oligonucleotides that contain subsequences that bind different primers.

Invariably, there is some precipitation or settling of components in a hybridization buffer during storage. Thus, in those embodiments of the present invention that include a hybridization buffer, its components are re-suspended, typically by heating and mixing, prior to use. The reagent is assembled (if not supplied as such in the kit) then added to the hybridization buffer. The resultant mixture is added to the microarray, which is then covered and incubated under suitable conditions to allow the nucleic acid binding events to occur. In those cases wherein the detectable label is a fluorescent dye, it is important that the array is stored in the dark until scanned. The fluorescence of dyes, particularly Cy5, diminishes rapidly even in ambient light. Following incubation, the microarray is washed with another buffer solution to remove non-bound reagents. The microarray is then scanned in accordance with standard procedures. In preferred embodiments of the present invention wherein the detectable labels are the fluorescent dyes Cy3™ and Cy5™, which are scanned via dual channel analysis, it is preferred that both channels are scanned simultaneously or that the Cy5™ channel is scanned first, followed by the Cy3™ channel.

Standard procedures e.g., for preparing the nucleic acids, spotting the nucleic acids onto the microarrays, and scanning the microarrays, typically entail on or more of the following: preparation of total RNA from cultured human cells; preparation of polyA+mRNA from total human RNA; amplification and purification of cDNAs for microarray manufacture; microarray manufacture and processing; generating control mRNAs by in vitro transcription; generating fluorescent cDNA controls by linear PCR; preparation of fluorescent probes from total human mRNA; cDNA microarray hybridization and washing; gene expression analysis with microarrays; and mutation detection with oligonucleotide microarrays. These procedures are described in M. Schena and R. W. Davis (1998). Genes, Genomes and Chips. In DNA Microarrays: A Practical Approach (ed. M. Schena), Oxford University Press, Oxford, UK, in press; Schena, M. and R. W. Davis (1998). Parallel Analysis with Biological Chips. in PCR Methods Manual (eds. M. Innis, D. Gelfand, J. Sninsky), Academic Press, San Diego, in press; Lemieux, B., Aharoni, A., and M. Schena (1998). Overview of DNA Chip Technology. Molecular Breeding 4, 277-289; Schena, M., Heller, R. A., Theriault, T. P., Konrad, K., Lachenmeier, E., and R. W. Davis (1998). Microarrays: biotechnology's discovery platform for functional genomics. Trends in Biotechnology 16:301-306; Heller, R. A., Schena, M., Chai, A., Shalon, D., Bedilion, T., Gilmore, J., Woolley, D. E., and Davis, R. W. (1997); Discovery and analysis of inflammatory disease-related genes using cDNA microarrays. Proceedings of the National Academy of Sciences USA 94:2150-2155; Schena, M., Shalon, D., Heller, R., Chai, A., Brown, P. O., and R. W. Davis. (1996). Parallel Human Genome Analysis: Microarray-Based Expression Monitoring of 1,000 Genes. Proceedings of the National Academy of Sciences USA 93: 10614-10619; Schena, M. (1996). Genome analysis with gene expression microarrays. BioEssays 18:427-431; Schena, M., Shalon, D., Davis, R. W. and Brown, P. O. (1995). Quantitative monitoring of gene expression patterns with a complementary DNA microarray. Science 270:467-470; Vishwanath, et al., Science 283:83-87 (1999); Nilsen, et al., J. Theor. Biol. 187:273-284 (1997); Sambrook, et al., (Eds.), Molecular Cloning, A Laboratory Manual (2nd Ed.), Cold Spring Harbor Laboratory Press (1989); and Ausubel, et al., (Eds.), Current Protocols in Molecular Biology, John Wiley & Sons, Inc. (1998).

The following examples are intended to further illustrate certain preferred embodiments of the invention and are not limiting in nature. Unless indicated otherwise, all parts and percentages are by weight.

### EXAMPLE 1

### Method for Detection and Quality Control Using a Random Oligonucleotide Labeled DNA Matrix A Detection Kit for cDNA Arrays

Kit Contents:

Vial 1 Random Sequence Cy3® 3DNA® Reagent (Genisphere, Montvale, N.J.). Use at 2.5 μL per 20 μL assay.

Vial 2 Hybridization buffer—0.25 M NaPO₄, 4.5% SDS, 1 mM EDTA, and 1×SSC. (Stored at −20° C. in the dark.)

Microarray Preparation:

A microarray was prepared as directed by the manufacturer or by customary protocol procedures. The nucleic acid sequences containing the DNA or gene probes were amplified using known techniques in polymerase chain reaction (PCR), then spotted onto glass slides, and processed according to conventional procedures.

3DNA® Reagent Preparation:

The Cy3® 3DNA® reagent is schematically illustrated in FIG. 3. The reagent 30 was prepared as follows. Oligonucleotide 31 having the general structure outlined below was synthesized. 5′-NNNNNNNNN-Matrix Sequence Complement-3′, wherein N represents a random nucleotide.

Matrix Sequence Complement 33 is an oligonucleotide sequence that hybridizes to outer surface arms 35 of matrix 37. This oligonucleotide was hybridized and cross-linked to DNA matrices that were also labeled with about 250 Cy3 oligonucleotides 39.

3DNA® Array Hybridization:

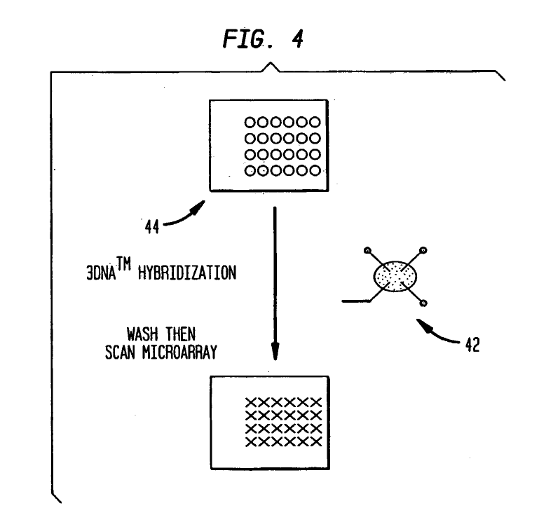

The hybridization buffer of Vial 2 was thawed and resuspended by heating to 65° C. for 10 minutes. The buffer was mixed by inversion to ensure that the components were resuspended evenly. If necessary, the heating and mixing were repeated until all the components were resuspended. Two- and one-half (2.5) μL of 3DNA® reagent of Vial 1 were added to 17.5 μL of hybridization buffer to yield a hybridization mixture. As schematically illustrated in FIG. 4, the hybridization mixture including Cy3® 3DNA® reagent 42 was added to microarray 44. The microarray was covered and incubated at a temperature of from about 37° C. to 42° C. for about 2-6 hours to overnight in a humidified chamber.

Post-Hybridization Wash:

The microarray was washed for 10 minutes at 42° C. with 2×SSC buffer containing 0.2% SDS. The microarray was then washed for 10 minutes at room temperature with 2×SSC buffer. The microarray was then washed for 10 minutes at room temperature with 0.2×SSC buffer.

Signal Detection:

The microarray was then scanned as directed by the scanner's manufacturer for detecting, analyzing, and assaying the hybridization pattern.

### EXAMPLE 2

### Method for Detection and Quality Control using a Primer-Specific Binding DNA Matrix A Detection Kit for cDNA Arrays

Kit Contents:

Vial 1 Primer Specific Binding Cy3® 3DNA® Reagent (Genisphere, Montvale, N.J.). Use at 2.5 μL per 20 μL assay.

Vial 2 Hybridization buffer—0.25 M NaPO₄, 4.5% SDS, 1 mM EDTA, and 1×SSC. (Stored at −20° C. in the dark.)

Microarray Preparation:

A microarray was prepared as directed by the manufacturer or by customary protocol procedures. The nucleic acid sequences comprising the DNA or gene probes were amplified using known techniques in PCR, then spotted onto glass slides, and processed according to conventional procedures.

3DNA® Reagent Preparation:

Oligonucleotides,

| | |
|---|---|
| 5′ ATT TAG GTG AGA CTA TAT TTT GG-3′ | (SEQ ID NO:1) = SP6-7BO |
| 5′ TAA TAC GAC TCA CTA TAG GGT TTT TCG-3′ | (SEQ ID NO:2) = T7-7BO |
| 5′ TAA CCC TCA CTA AAG GGA TTT TTC-3′ | (SEQ ID NO:3) = T3-7BO |
| 5′ GTT GTA AAA CGA CCA GTG TTT TTCG-3′ | (SEQ ID NO:4) = M13Forward-7BO |
| 5′ CAC ACA GGA AAC AGC TAT GTT TTT GG-3′ | (SEQ ID NO:5) = M13Reverse-7BO, |

were synthesized by an outside vendor (Oligos Etc, Inc. Wilsonville, Oreg.), and ligated to the outer arms of a Cy3 labeled DNA matrix.

3DNA® Array Hybridization:

The hybridization buffer of Vial 2 was thawed and re-suspended by heating to 65° C. for 10 minutes. The buffer was mixed by inversion to ensure that the components were re-suspended evenly. If necessary, the heating and mixing were repeated until all the components were re-suspended. Two and one-half (2.5) μL of 3DNA® reagent of Vial 1 were added to 17.5 μL of hybridization buffer to yield a hybridization mixture. The hybridization mixture was added to the microarray. The microarray was covered and incubated at a temperature of from about 37 to 42° C. for about 2-6 hours to overnight in a humidified chamber.

Post-Hybridization Wash:

The microarray was washed for 10 minutes at 42° C. with 2×SSC buffer containing 0.2% SDS. The microarray was then washed for 10 minutes at room temperature with 2×SSC buffer. The microarray was then washed for 10 minutes at room temperature with 0.2×SSC buffer.

Signal Detection:

The microarray was then scanned as directed by the scanner's manufacturer for detecting, analyzing, and assaying the hybridization pattern.

### EXAMPLE #3

### Method for Detection and Quality Control using a Random Oligonucleotide with a Capture Sequence and a Cy3 Labeled DNA Matrix A Detection Kit for cDNA Arrays

Kit Contents:

Vial 1 Cy3® 3DNA® Reagent (Genisphere, Montvale, N.J.). Use at 2.5 μL per 20 μL assay.

Vial 2 Random Sequence Oligonucleotide with 3DNA capture sequence

Vial 2 Hybridization buffer—0.25 M NaPO₄, 4.5% SDS, 1 mM EDTA, and 1×SSC. (Stored at −20° C. in the dark.)

Microarray Preparation:

A microarray was prepared as directed by the manufacturer or by customary protocol procedures. The nucleic acid sequences containing the DNA or gene probes were amplified using known techniques in PCR, then spotted onto glass slides, and processed according to conventional procedures.

3DNA® Reagent Preparation:

An oligonucleotide having the general structure outlined below was synthesized.

(SEQ ID NO:6)

5′-GGC CTC ACT GCG CGT CTT CTG TCC CGC CTT TTT CG-

3′

### | - - - Matrix Capture Sequence Complement - - - |

This oligonucleotide was ligated to a Cy3 labeled matrix. The matrix capture sequence complement is an oligonucleotide sequence that hybridizes to the 5′ end of a bifunctional oligonucleotide (contained in vial #2), one end of which binds to sequences spotted on a microarray, in this case random sequences, and a second end which hybridizes to the complementary sequence attached to the matrix.

Random sequence oligonucleotide with 3DNA capture sequence:

An oligonucleotide having the general structure outlined below was synthesized.

5′-NNNNNNNNN-Matrix Capture Sequence-3′

3DNA® Array Hybridization:

The hybridization buffer of Vial 2 was thawed and re-suspended by heating to 65° C. for 10 minutes. The buffer was mixed by inversion to ensure that the components were re-suspended evenly. If necessary, the heating and mixing were repeated until all the components were re-suspended. Two and one-half (2.5) μL of 3DNA® reagent of Vial 1 were added to 17.5 μL of hybridization buffer to yield a hybridization mixture. The hybridization mixture was added to the microarray. The microarray was covered and incubated at a temperature of from about 37 to 42° C. for about 2-6 hours to overnight in a humidified chamber.

Post-Hybridization Wash:

The microarray was washed for 10 minutes at 42° C. with 2×SSC buffer containing 0.2% SDS. The microarray was then washed for 10 minutes at room temperature with 2×SSC buffer. The microarray was then washed for 10 minutes at room temperature with 0.2×SSC buffer.

Signal Detection:.

The microarray was then scanned as directed by the scanner's manufacturer for detecting, analyzing, and assaying the hybridization pattern.

### Industrial Applicability

The invention is useful in the field of diagnostics, particularly as it pertains to screening individuals

All patent and non-patent publications cited in this specification are indicative of the level of skill of those skilled in the art to which this invention pertains. All these publications and patent applications are herein incorporated by reference to the same extent as if each individual publication or patent application was specifically and individually indicated as being incorporated by reference herein.

Those skilled in the art will recognize, or be able to ascertain, using no more than routine experimentation, numerous equivalents to the specific substances and procedures described herein. Such equivalents are considered to be within the scope of this invention, and are covered by the following claims.

1. A kit for conducting quality control reactions on a microarray of nucleic acids, comprising:
a container comprising a first buffer solution comprising a first reagent comprising a nucleic acid matrix carrying a detectable label, said matrix having attached thereto an oligonucleotide that binds nucleic acid contained on any position on the microarray; and
directions for conducting the quality control reactions with said first reagent and the nucleic acids on the microarray.

2. The kit of claim 1 wherein said matrix comprises a polynucleotide monomer comprising an intermediate region comprising a linear, double stranded waist region having a first end and a second end, said first end terminating with two single stranded hybridization regions, each from one strand of the waist region, and said second end terminating with one or two single stranded hybridization regions, each from one strand of the waist region.

3. The kit of claim 2 wherein each of said hybridization regions and said waist region of said monomer comprise sequences obtained from a master sequence containing no repeats of subsequences having X nucleotides wherein X represents an integer of from 2 to 6.

4. The kit of claim 1 wherein said matrix comprises a plurality of polynucleotide monomers bonded together by hybridization; each polynucleotide monomer having an intermediate region comprising a linear, double stranded waist region having a first end and a second end, said first end terminating with two single stranded hybridization regions, each from one strand of the waist region, and said second end terminating with one or two single stranded hybridization regions, each from one strand of the waist region; and in said polynucleotide each polynucleotide monomer is hybridization bonded to at least one other polynucleotide monomer at at least one such hybridization region.

5. The kit of claim 4 wherein each of said hybridization regions and said waist regions of said plurality of monomers comprise sequences containing no repeats of subsequences having X nucleotides, wherein X represents an integer of at least 2.

6. The kit of claim 1 wherein said oligonucleotide has a random sequence.

7. The kit of claim 1 wherein said oligonucleotide binds a primer sequence selected from the group consisting of T7, T3, M13 forward, M13 reverse and SP6.

8. The kit of claim 1 wherein said oligonucleotide is attached to said matrix via ligation.

9. The kit of claim 1 wherein said oligonucleotide is attached to said matrix via hybridization and cross-linking.

10. The kit of claim 1 wherein said detectable label is a fluorescent dye.

11. The kit of claim 10 wherein said fluorescent dye is Cy3™ or Cy5™.

12. The kit of claim 10 wherein said fluorescent dye is Alexa™ 488 or Alexa™ 594.

13. The kit of claim 1 further comprising a second container comprising a second buffer solution for conducting the quality control reactions with said reagent and the nucleic acids on the microarray.

14. The kit of claim 1 further comprising a second container comprising a second buffer solution containing a second reagent comprising a nucleic acid matrix carrying a detectable label, said matrix having attached thereto an oligonucleotide that binds nucleic acid contained on the microarray, wherein the detectable label of the first reagent and the detectable label on the second reagent are resolvable from each other; and
wherein said directions explain how to use said first and second reagents with the microarray.

15. The kit of claim 14 wherein the oligonucleotide attached to said first reagent and the oligonucleotide attached to said second reagent bind different nucleic acids on the microarray.

16. A kit for conducting quality control reactions on a microarray of nucleic acids, comprising:
a first container comprising a first buffer solution comprising a nucleic acid matrix carrying a detectable label; and
directions for producing a reagent by attaching to said matrix an oligonucleotide probe having a first end portion attachable to said matrix and a second end portion that binds nucleic acid on any position on the microarray, and conducting the quality control reactions with the reagent and the nucleic acids on the microarray.

17. The kit of claim 16 further comprising a second container containing a second buffer solution in which to conduct the quality control reactions between the reagent and the nucleic acids on the microarray.

18. A kit for conducting quality control reactions on a microarray of nucleic acids, comprising:
a first container comprising a first buffer solution comprising a nucleic acid matrix carrying a detectable label;
a second container comprising a second buffer solution comprising an oligonucleotide probe having a first end portion attachable to said matrix and a second end portion that binds nucleic acid on any position on the microarray; and
directions for attaching said oligonucleotide probe to said matrix to prepare to first reagent and for conducting the quality control reactions with the first reagent and the nucleic acids on the microarray.

19. The kit of claim 18 further comprising a third container containing a third buffer solution in which to attach said oligonucleotide probe to said matrix.

20. The kit of claim 18 wherein said matrix has attached thereto a complement capture oligonucleotide and wherein said oligonucleotide probe attaches to said matrix via hybridization and cross-linking to said complement capture oligonucleotide.

21. A method for preparing a kit for conducting quality control reactions on a microarray of nucleic acids, comprising:
providing a container comprising a buffer solution comprising a reagent comprising a nucleic acid matrix carrying a detectable label, said matrix having attached thereto an oligonucleotide that binds nucleic acid contained on any position on the microarray;
providing directions for conducting the quality control reactions with said reagent and the nucleic acids on the microarray; and
packaging the container and the directions in the form of a kit.

22. A method for preparing a kit for conducting quality control reactions on a microarray of nucleic acids, comprising:
providing a container comprising a buffer solution comprising a nucleic acid matrix carrying a detectable label;
providing directions for preparing a reagent by attaching to said matrix an oligonucleotide having a first end portion attachable to said matrix and a second end portion that binds nucleic acid on the microarray, and conducting the quality control reactions with the reagent and the nucleic acids on any position on the microarray; and
packaging the first container and the directions in the form of a kit.

23. A method for preparing a kit for conducting quality control reactions on a microarray of nucleic acids, comprising:
providing a first container comprising a first buffer solution comprising a nucleic acid matrix carrying a detectable label;
providing a second container comprising a second buffer solution comprising an oligonucleotide having a first end portion attachable to said matrix and a second end portion that binds nucleic acid on any position on the microarray;
providing directions for attaching said oligonucleotide to said matrix to prepare a reagent and for conducting the quality control reactions with the reagent and the nucleic acids on the microarray; and
packaging the first container, the second container and the directions in the form of a kit.

24. A method for conducting quality control reactions on a microarray of nucleic acids, comprising:
providing the microarray of nucleic acids;
providing a reagent comprising a nucleic acid matrix carrying a detectable label, said matrix having attached thereto an oligonucleotide that binds nucleic acid contained on any position on the microarray;
contacting the reagent with the microarray; and
detecting the label as an indication of presence or type of nucleic acid on the microarray.

25. A method for conducting quality control reactions on a microarray of nucleic acids, comprising:
providing a nucleic acid matrix carrying a detectable label and an oligonucleotide probe having a first end portion attachable to said matrix and a second end portion that binds nucleic acid on the microarray;
preparing a reagent by attaching the oligonucleotide probe to said matrix;
contacting the reagent with the microarray; and
detecting the label as an indication of presence or type of nucleic acid on the microarray.
